(19) United States
(12) Patent Application Publication
(10) Pub. No.: US 2018/0325748 A1
(43) Pub. Date: Nov. 15, 2018

(54) CELLULOSE ACETATE BASED NON-WOVEN NANOFIBER MATRIX WITH HIGH ABSORBENCY PROPERTIES FOR FEMALE HYGIENE PRODUCTS

(71) Applicants: CHANDRA SHEKHAR SHARMA, MEDA DISTRICT, TELANGANA (IN); SHITAL YADAV, MEDAK DISTRICT, TELANGANA (IN); TULIKA RASTOGI, LUCKNOW (IN); ILA MANI PUJITHA, YEDUMAILARAM, MEDAK DISTRICT (IN)

(72) Inventors: CHANDRA SHEKHAR SHARMA, MEDA DISTRICT, TELANGANA (IN); SHITAL YADAV, MEDAK DISTRICT, TELANGANA (IN); TULIKA RASTOGI, LUCKNOW (IN); ILA MANI PUJITHA, YEDUMAILARAM, MEDAK DISTRICT (IN)

(21) Appl. No.: 15/329,583

(22) PCT Filed: Jul. 27, 2015
(86) PCT No.: PCT/IB2015/001264

§ 371 Date: Jan. 30, 2017

(30) Foreign Application Priority Data
Jul. 28, 2014 (IN) 3684/CHE/2014

(51) Int. Cl. A61F 13/53; A61F 13/472; A61F 13/15; D04H 1/425; D04H 1/728

(52) U.S. Cl. CPC A61F 13/53; A61F 13/472; A61F 13/15804; A61F 13/15252; D10B 2509/026; D04H 1/728; A61F 2013/530043; D10B 2201/28; D04H 1/425

(57) ABSTRACT

The main objective of the present invention is to demonstrate a biocompatible polymer matrix in feminine hygiene products. Another objective of the invention is to produce the biocompatible polymer matrix in the form of non-woven nanofibers so as to enhance the properties such as surface area, absorption rate, tensile strength etc. Yet another objective of the present invention is to study the effect of SAP on the absorpotion capacity of absorbent matrix prepared as mentioned above. Accordingly, the present invention discloses an eco-friendly sanitary napkin characterized with absorbancy core having enhanced properties like absorbancy, tensile strength etc., without addition of SAP.

### FIELD OF THE INVENTION

The present invention relates to an eco-friendly female hygienic product made up of biocompatible polymer nanofibers more particularly to cellulose acetate nanofibers electrospun with and without super-absorbent polymer into its non-woven fiber matrix. This invention replaces these microfibers with nanofibers, thereby achieving higher surface area to volume ratio and tunable porosity, resulting in enhanced properties of these fibers like absorbency rate and reduced residual percentage.

### BACKGROUND OF THE INVENTION

Menstrual hygiene is an important issue for every woman, as poor menstrual hygiene increases the vulnerability towards reproductive tract infections (RTIs) [1]. There are different types of feminine hygiene products commercially available such as sanitary napkins, tampons, panty shields, wipes and cosmetic removal pads. Among these, feminine sanitary pad/napkin is an important disposable absorbent hygiene product. Its functions are to absorb and retain menstrual fluid discharge and isolate it from skin, along with maintaining comfort, preventing odor and staying in place [2]. To accomplish all these requirements, sanitary pads constitutes different layers like cover stock, acquisition and distribution layer, absorbent core, back sheet, tissue, elastic wing and siliconized paper [2]. Absorbent core gives the desired absorption capacity to sanitary pads and is mainly made up of hydrophilic cellulosic fibers such as wood derived fluff pulp or viscose rayon [2]. As the diameter of these cellulosic fibers present in commercially available products is in range of few tens of microns, their absorption capacity is less owing to their lower surface area. To improve the absorption capacity, some of the commercially available female hygiene products use superabsorbent polymers (SAPs), either in the form of granules within cellulosic fiber matrix or in the form of composite fabric layer [2].

SAPs are commonly divided into two main classes i.e., synthetic (petrochemical-based) and natural (polysaccharide- and polypeptide based) [3]. Most of these SAPs are produced from acrylic acid, its salt and acrylamide [3]. The superabsorbents available in the market today are primarily based on cross-linked sodium polyacrylate (SPA) gels [2]. It facilitates in increasing the liquid absorption capacity and liquid retention capacity tremendously, thus allowing the product to be thinner but with improved performance [2].

However, there are some harmful chemicals present in the commercially available sanitary napkins. For example, dioxins are used to bleach the material used for making absorbent core, especially cotton, but it causes side effects in the body such as pelvic inflammatory disease, ovarian cancer, immune system damage, impaired fertility, diabetes, etc. [4]. As mentioned above, SAPs are added to increase the absorption capacity, but in 1980s, use of SAPs is restricted in tampons due to its possible link with toxic shock syndrome, potentially fatal illness caused by a bacterial toxin [5]. Further as SAPs are petroleum based products and therefore does not degrade readily in landfills, their use is not eco-friendly as well.

US20090012487 entitled “Sanitary napkin containing herb ingredients” discloses a sanitary napkin that contains polymeric absorbents and herbal ingredients, particularly to a functional sanitary napkin, in which an absorbent layer structure, having the polymeric absorbents and herbal ingredients distributed thereon consisting of three layers of non-woven fabric and surrounded by a polymeric absorbent, containing surge layer made of an air-laid material, such that the ability to absorb menstrual blood is augmented and the odor of the herbal ingredients is prevented from permeating through undergarments, garments and the like. Even though this product used herbal ingredients for the sanitary napkin to make it natural and easy for disposal, the use of polymeric absorbant which is nothing but SAP causes health effects as mentioned above.

Therefore the objective of the present invention is to minimize the use of SAPs in female hygiene products considering their possible adverse health effects. To achieve this, the present invention discloses a bio-compatible sanitary napkin wherein cellulose based nanofibers are fabricated and used as absorbent core. The increased specific surface area of nanofibers as compared to micron sized fibers present in commercial products also justifies well this objective and may compensate for the absorption capacity while using SAPs.

Electrospinning is one of the simple and cost effective method used to synthesize fibers with diameter ranging from 10 nm to 10 μm [6,7]. This method is invented by Formhals in 1934 [8]. Electrospinning process uses high electric field as a driving force to draw fibers from electrically charged polymer solution or polymer melt [6-10]. Electrospun fibers possess certain enhanced characteristics such as high surface-to-volume ratio, tunable porosity and flexible morphology with controllable diameter [11], making them suitable for use in wide range of applications.

Apart from the health problems attributed to the synthetic sanitary napkins available in the market, there are other limitations found, such as: in few products, cellulosic derivatives such as rayon and viscous fibers, are treated to add fragrance or to enhance appearance by bleaching which leads to side effects on health; micro fibers prepared from ionic liquids have low absorption capacity [12]; if SAPs are used to increase the absorption capacity of Rayon and viscous fibers, then probability of toxic shock syndrome increases, which in turn might lead to potentially fatal illness caused by a bacterial toxin; when liquid comes in contact with SAPs, they start swelling due to absorption of liquid, and as the percentage absorption increases, SAPs get sticky in nature and can attach to skin causing skin irritation; distribution of SAP granules inside the absorbent core is also irregular as these granules come out of the absorbent core on absorbing liquid; and disposal of used sanitary products by either flushing out into the oceans, incinerating or depositing in landfill creates various pollutants as they are neither biodegradable nor recyclable.

The main objective of the present invention is to exploit the large surface area of electrospun nanofibers in achieving the high absorption capacity. Cellulosic fibers have been used for absorption of water and other aqueous fluids. However solvents used for cellulose, such as ionic liquids, are not completely volatile and require coagulation step to get stable fibers. On the other hand, cellulose derivatives such as cellulose acetate, hydroxypropyl cellulose, hydroxypropyl methyl cellulose etc. can be easily dissolved in different volatile solvents that make them suitable for electrospinning. Among these derivatives, cellulose acetate is biocompatible, biopolymer which is easily available and has low cost [13]. It shows good hydrolytic stability and can be recycled in environment by biodegradation [14]. Therefore, cellulose acetate is chosen as a material to prepare nanofabric matrix for its use as an absorbent core.

Accordingly, the present invention discloses a biocompatible sanitary napkin comprising of electrospun nanofibers of cellulose acetate are fabricated and characterized in terms of its surface morphology and mechanical properties. To demonstrate its use in female hygiene application, different tests such as free absorbency, equilibrium absorbency, absorbency under load and percentage residue are performed in different mediums i.e., distilled water, saline solution and synthetic urine respectively and the results obtained are then compared with some of the known commercially available feminine sanitary napkins.

### SUMMARY OF THE INVENTION

The main objective of the present invention is to demonstrate a biocompatible polymer matrix in feminine hygiene products. Another objective of the invention is to produce the biocompatible polymer matrix in the form of non-woven nanofibers so as to enhance the properties such as surface area, absorption rate, tensile strength etc. Yet another objective of the present invention is to study the effect of SAP on the absorpotion capacity of absorbent matrix prepared as mentioned above.

Surface morphology and specific surface area for this electrospun cellulose acetate nanofibrous mat with and without SPA are studied using Field Emission Scanning Electron Microscopy (FESEM) and Brunauer-Emmett-Teller (BET) adsorption method. Further to investigate their absorbent properties, free absorbency at different time intervals and equilibrium absorbency are measured in distilled water, saline solution (0.9 wt. % NaCl) and synthetic urine respectively. Absorbency under load is also tested in saline solution for practical use. The amount of residue and tensile properties was determined for these electrospun CA based nanofibrous mats to enable their use as absorbent core in female hygiene products.

While comparing all these results with six different types of commercially available feminine sanitary napkins which are primarily composed of micron sized cellulosic fibers with superabsorbent polymers in the form of granules or fabric, it is found that pure CA electrospun nanofibers shows significantly higher absorbency in all conditions in all different mediums used. Hence the use of electrospun CA nanofibers in place of micron size fabric in commercial female sanitary napkins not only enhances the absorption properties, mechanical strength and remarkably reduces residual percentage but also eliminates the use of SAP without compromising its performance. This in turn may pave the way to resolve many health and environmental issues related with the use non-biodegradable SAP.

### BRIEF DESCRIPTION OF THE DRAWINGS

The above-mentioned description along with others advantages of this present disclosure, and the manner of attaining them, will become more apparent and the present disclosure will be better understood by reference to the following description of embodiments of the present disclosure taken in conjunction with the accompanying drawings, wherein:

FIG. 1 illustrates schematic representation of an electrospinning set-up 100 including a syringe pump 101, syringe 102, polymer solution 103, needle 104, collector 106 and power supply 105 among others;

FIG. 2(a) shows SEM image of commercial sample, S1; FIG. 2(b) shows FESEM image of electrospun fibers of SA; FIG. 2(c) shows FESEM image of electrospun fibers of SB; and FIG. 2(d) shows FESEM image of electrospun fibers of SC;

FIG. 3 shows the graphical representation of the percentage of free absorbancy against time, recorded for free absorbency test conducted in (a) distilled water (b) saline solution (c) synthetic urine and (d) equilibrium absorbency respectively for all samples such as SA, S, SC, S1, S2, S3, S4, S5 and S6;

FIG. 4 shows the test setup used to determine absorbency under load wherein it comprises of a glass filter plate 205, petri dish 206, cylindrical beaker 201 etc;

FIG. 5 shows a graphical representation of equilibrium absorbency and absorbency under load in saline solution for different electrospun nanofibers samples (SA, SB and SC) and selected commercial samples (S1 to S6);

FIG. 6 shows a pictorial representation of the absorbent cores of commercial samples (a) S1 (b) S2 (c) S5 (d) S4 and (e) electrospun CA nanofibers (SA, SB and SC) and the changes after dipping in distilled water for 10 minutes respectively;

FIG. 7 shows a representation of the residue percentage found in residue test conducted for electrospun fiber samples (SA and SB) and commercial samples (S1 and S2); and

FIG. 8 shows a representation of the Young's Modulus for different samples (electrospun SA nano fibers and commercial samples S1, S2, S4 and S5).

### DETAILED DESCRIPTION OF THE PREFERRED EMBODIMENTS

Reference will now be made in detail to the experiments conducted for the invention. Before describing the detailed experiments that are in accordance with the present disclosure, it should be observed that such experiments reside primarily in combinations of process/method steps and the product.

In this document, the terms “comprises,” “comprising,” or any other variation thereof, are intended to cover a non-exclusive inclusion, such that a process, product, method, article, device or apparatus that comprises a list of elements does not include only those elements but may include other elements not expressly listed or inherent to such process, product, method, article, device, or apparatus. An element proceeded by “comprise . . . a” does not, without more constraints, preclude the existence of additional identical elements in the process, product, method, article, device or apparatus that comprises the element.

Any embodiment described herein is not necessarily to be construed as preferred or advantageous over other embodiments. All of the embodiments described in this detailed description are illustrative, and provided to enable persons skilled in the art to make or use the disclosure and not to limit the scope of the disclosure, which is defined by the claims.

It will be apparent to those skilled in the art that various modifications and variations can be made to the present invention without departing from the spirit and scope of the invention. Thus it is intended that the present invention covers the modifications and variations of this invention provided they come within the scope of the appended claims and their equivalents.

In the following description, for the purpose of explanation, numerous specific details are set forth in order to provide a thorough understanding of the present product of biocompatible sanitary napkin and its characteristics. It will be apparent, however, to one skilled in the art that the present invention can be practiced without these specific details.

The present invention discloses an eco-friendly sanitary napkin comprising a biocompatible polymer matrix in the form of non-woven nanofibers which has certain enhanced properties such as higher surface area, absorbancy, tensile strength and does not give any side effects to the health. The below experiments are conducted to showcase the resulted properties of the product and to prove that the addition of SAPs in the female hygiene product will actually reduce the absorbancy. To prove the enhanced properties of the product disclosed in the present invention, a comparison study is done by conducting experiments on the above mentioned product and other commercially available sanitary napkins.

### Preparation of Sample and Other Required Material:

Cellulose acetate (Mₙ, 29,000) and poly (acrylic acid sodium salt) (Mₙ, 5,100) are purchased from Sigma-Aldrich, India. Acetone (99% purity) and N,N-dimethylacetamide (99.5%) are received from Merck India. Distilled water from Millipore is used throughout the experiments.

### Commercial Feminine Sanitary Napkins as Reference:

There are several different types of disposable menstrual pads available in the market. They are classified on basis of their use in different conditions. Different commercial products (S1-S6) of various categories considered and used in the comparison study of this invention are summarized in Table 1, below.

TABLE 1

| Sample | Commercial | | |
|---|---|---|---|
| Reference | Product Name | Category | Conditions of use |
| S1 | Whisper Ultra Clean | Ultra-thin | Heavy flow |
| S2 | Whisper Choice Ultra | Regular | Low to |
| | | | medium flow |
| S3 | Stayfree Secure | Regular | Low to |
| | (Regular) | | medium flow |
| S4 | Whisper Maxi-fit | Maxi/Super | Heavy flow |
| S5 | Whisper Maxi | Overnight/ | Very heavy |
| | nights | Maternity | flow |
| S6 | Carefree Panty Liner | Panty Liner | Daily flow |

### Synthetic Urine Preparation:

Synthetic urine is prepared by adding the following to distilled water to give a solution with a final volume of 1 litre: 25 g urea, 9 g sodium chloride, 2.5 g sodium phosphate, 3 g ammonium chloride, and 3 g sodium sulfite [15].

Polymer Solution Preparation with and without SAP:

Cellulose acetate is dissolved in a mixture of acetone and N,N-dimethylacetamide (DMA) (2:1, v:v) to make 16 wt. % solution for electrospinning. The mixture is stirred to get a clear and transparent solution of cellulose acetate. In two other formulations, 5% (w/v) and 10% (w/v) solutions of sodium poly acrylate (SPA) are prepared by mixing SPA in methanol and then added to the above prepared cellulose acetate solution in 1:1 ratio. On adding SPA directly to cellulose acetate solution, it agglomerates and therefore is not recommended for electrospinning.

### Process of Preparation of Biocompatible Matrix—Electrospinning:

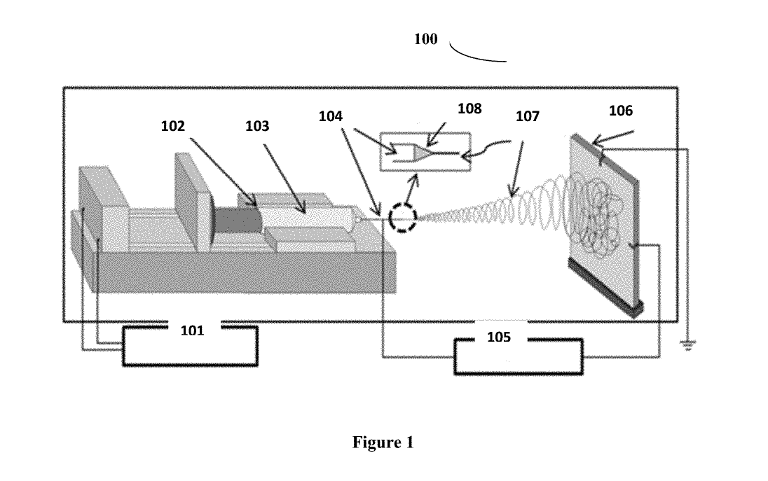

FIG. 1 illustrates the schematic of basic set up of the electrospinning process 100. Electrospinning process uses high electric field as a driving force to draw fibers from electrically charged polymer solution or polymer melt. The basic setup of electrospinning comprises of a syringe pump 101, voltage source 105 and a collector 106. Syringe pumps 101 helps in maintaining the desired flow rate. When sufficiently high voltage is applied to a liquid droplet, it becomes charged and electrostatic repulsion counteracts the surface tension, resulting in change in shape of the droplet, known as the Taylor cone 108. At this point, liquid erupts from the surface and fibers get deposited on the grounded collector 106. Polymer solution 103 gets charged and internal repulsion led to instability in polymer jet 107, Rayleigh instability or whipping motion of the jet, depending on electric field strength. Solvent evaporates in the distance between tip of the needle 104 and collector 106 and solidified deposition obtained on the collector. Electrospinning parameters such as feed rate, applied voltage, tip-to-collector distance and needle (tip) diameter need to be optimized to get continuous uniform nanofibers of desired morphology.

In this study, three different polymer solutions i.e., cellulose acetate (CA), cellulose acetate with 5 wt. % SPA (CA5) and cellulose acetate with 10 wt. % SPA (CA10), are used for electrospinning. Aluminum foil placed on the copper collector is used as a substrate to collect these electrospun fibers. The below given table summarizes the final parameters optimized for preparing different samples of cellulose acetate nanofibers as mentioned above by electrospinning wherein ‘SA’ represents cellulose acetate solution and ‘SB’ and ‘SC’ represent 5% (w/v) and 10% (w/v) solutions of SPA at 1:1 ratio respectively.

TABLE 2

| | | | Needle | |
|---|---|---|---|---|
| | Applied | Flow rate | diameter | |
| Sample | Voltage (kV) | (μl/minutes) | (gauge) | Distance (cm) |
| SA | 10 | 5 | 18 | 10 |
| SB | 12 | 10 | 18 | 10 |
| SC | 12 | 10 | 18 | 10 |

### Example 1

### Morphological Characterization

The surface morphologies of the electrospun nanofibers are observed using field emission scanning electron microscopy (FESEM) (Carl Zeiss, SUPRA 40). Electrospun nanofibers are removed from the aluminum foil and cut into small pieces of 1×1 cm². All samples SA, SB and SC are sputtered with thin layer of gold before image analysis in FESEM in order to minimize the charge effect. For commercial products considered as reference such as samples S1 to S6 (refer Table 1), absorbent core is removed and then examined in scanning electron microscope.

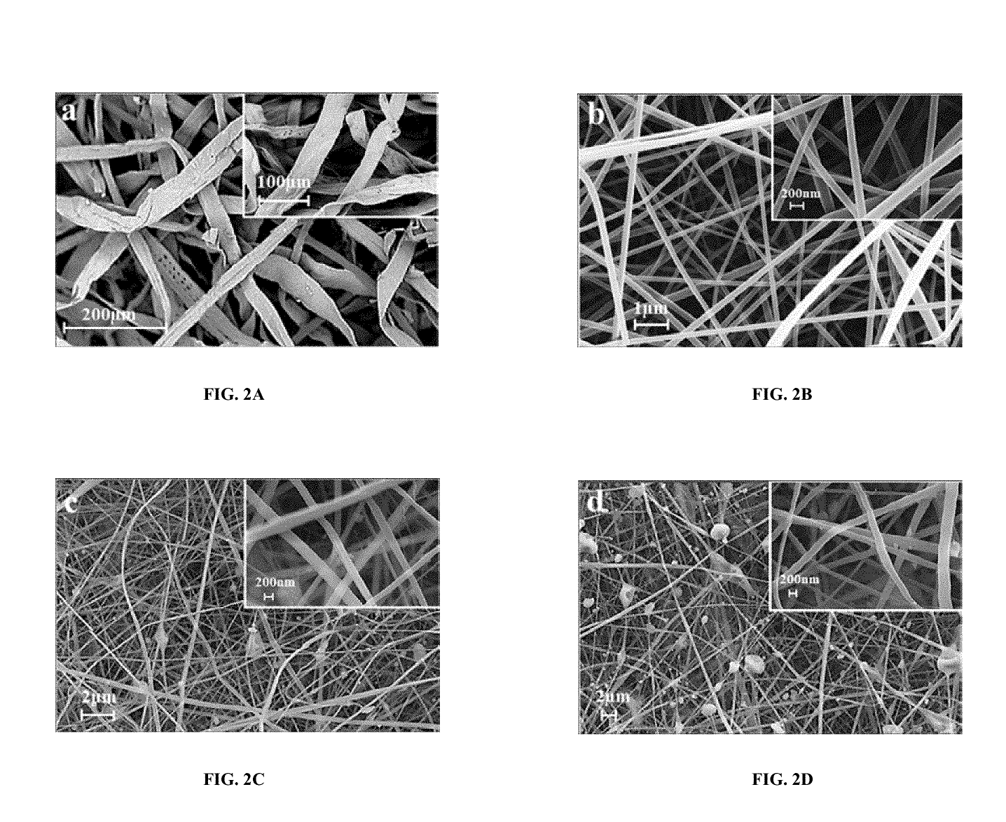

Surface morphology of absorbent core of selected commercial feminine sanitary napkins is examined using SEM. A representative SEM image of these fibers for sample S1 is shown here as FIG. 2a. Feminine sanitary napkins are made up of cellulosic fibers which are found to be in flat-ribbon like shape with width of about 40-50 μm.

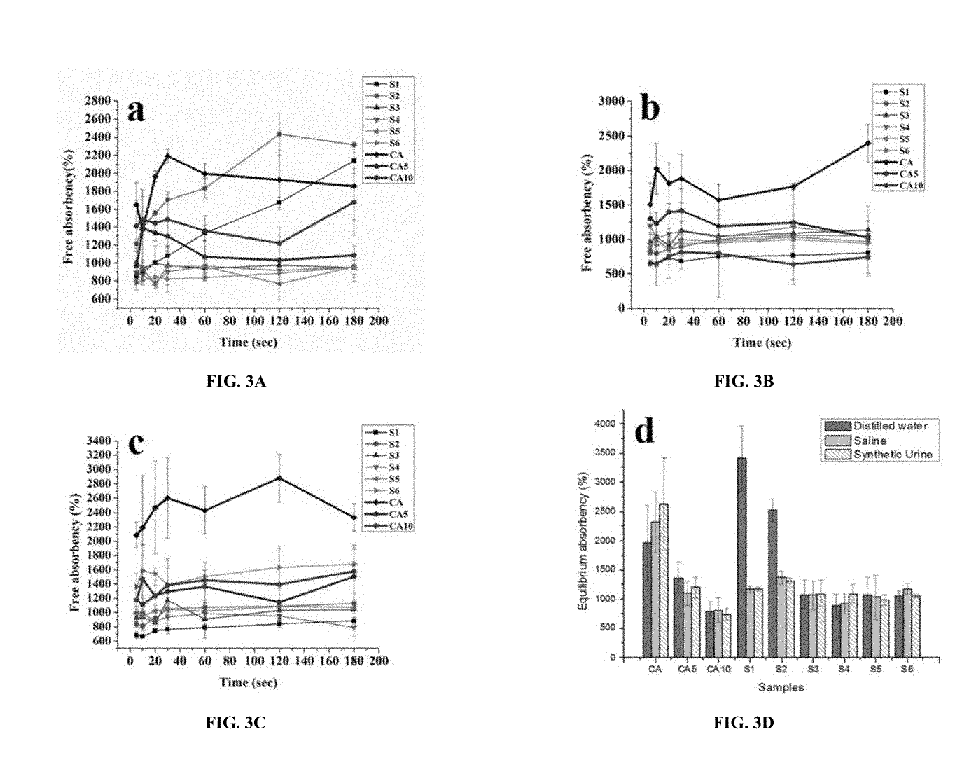

Electrospun CA nanofibers (SA) as shown in FIG. 2b are long, continuous, and uniform with diameter in the range of 50-150 nm. Solution of cellulose acetate with 5 wt. % of SPA (SB) is in suspension and its effect can be observed in the form of partially beaded fibers in FIG. 2c. Number of beaded fibers increased on increasing the SPA concentration to 10 wt. % (SC) as shown in FIG. 3d. However in both cases, fibers obtained are long and continuous as similar to only CA fibers. Fiber diameter for both the samples (SB and SC) is measured to be in range of 50-200 nm. From FESEM image analysis, it is clearly observed that fiber diameter is reduced to more than two order of magnitude for electrospun fiber samples as synthesized in this work as compared to fabric used in commercial products.

### Example 2

### Specific Surface Area (SSA) Measurement

The Brunauer-Emmett-Teller (BET) surface area of electrospun CA nanofibers with and without SPA and two different types of commercial samples (S1 and S4) is determined by N₂ physisorption using Quantachrome instruments v3.01. The commercial samples for this test are selected depending on the form of SAP present in it. One for granular powder form and another for sandwich layer form of SAP. The weight of the sample is fixed to be 100 mg. All samples are degassed at 80° C. for 60 minutes in nitrogen. The SSAs are determined by a multi-point BET measurement with nitrogen as the adsorbate.

BET surface area of electrospun CA nanofibers (SA) is found to be 50.21 m²/g which decreased to 22.14 m²/g and 18.36 m²/g when SPA is added as 5 (SB) and 10 wt. % (SC) respectively. This decrease in surface area for SB and SC samples may be attributed mainly due to increased fiber diameter and change in morphology from bead free to beaded fibers on encapsulation of SPA. Surface area of two commercial samples, sample S1 and S4 is measured to be 6.41 and 13.37 m²/g respectively. As we observe that surface area for electrospun CA nanofibers is significantly large compared to all other samples considered.

### Example 3

### Free Absorbency Test

This test is done to quantify the absorption capacity of any sample with respect to time, when allowed to swell freely. Electrospun nanofibers are moved from the aluminum foil to prepare free standing fabric mat. Similarly, absorbent core is removed from commercial products. These are then cut into approximately 2×2 cm² size and weighed (W1—dry weight). The sample is then placed in a beaker containing distilled water and removed after 5 seconds. The excess water is allowed to drain off with the help of tissue paper, for 30 seconds. The sample is weighed again (W2—wet weight). This process is continued with measurements taken after immersion for 10, 20, 30, 60, 120 and 180 seconds respectively. Free absorbency can be calculated as below:

Q=[(W2−W1)/W1]*100

### Where:

Q=Percent free absorbency;
W1=Initial (dry) weight of the sample without absorbent core; and
W2=Final (wet) weight of the sample without absorbent core.

Similar procedure is followed to determine the free absorbency with 0.9 wt. % solution of sodium chloride i.e., saline solution and synthetic urine.

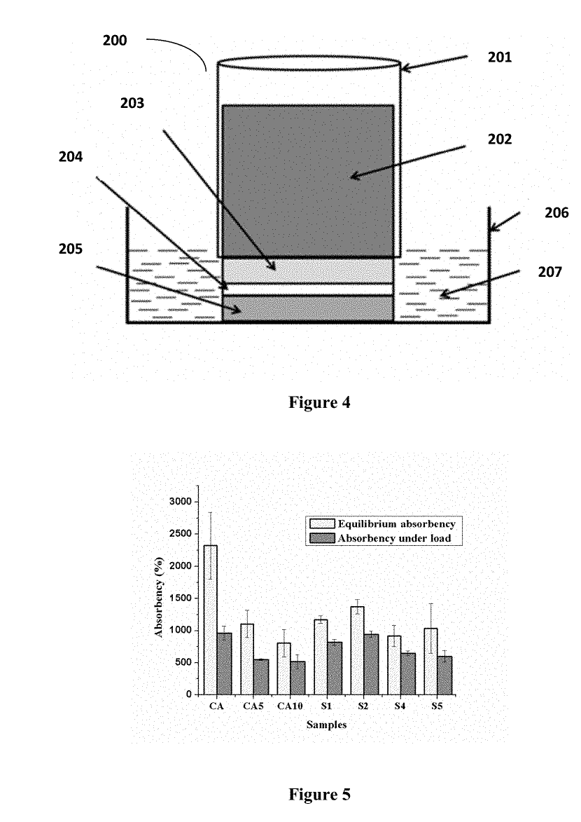

Free absorbency test is done using distilled water, saline solution and synthetic urine respectively to test the absorption capacity of samples. Percent absorbency of electrospun cellulose acetate nanofibers with and without SPA are measured and compared with the selected commercially available feminine sanitary napkins (FIG. 4a-c).

FIG. 3a represents the graphical representation of the free absorbency against time in distilled (DI) water. SPA is generally added to increase the absorption capacity and is found to achieve maximum swelling in DI water. However, its encapsulation in nanofibers not only restricts its swelling but interestingly decreases the absorption capacity of CA nanofibers in DI water. For small time span of 20 seconds, the percent absorbency of SA SB and SC nanofibers is measured to be 1963.1, 1336.4 and 1446.9% respectively. This shows that percent absorbency for pure CA nanofibers is 31.9 and 26.3% higher than SB and SC samples respectively. In spite of increasing the time interval to 180 seconds, pure CA nanofibers samples (SA) had 39.1% and 9.5% more absorbency as compared to SB and SC respectively. Therefore, CA nanofibers without SPA addition showed maximum percentage of free absorbency. When these results are compared with commercial samples taken as reference, in DI water, it is found that the absorption capacity of CA nanofibers for time interval of 20 seconds is about 48.6, 20.7, 49.2, 60.3, 61.3 and 57.1% higher than samples S1, S2, S3, S4, S5 and S6 respectively. When time interval is increased to 180 seconds, samples S1 and S2 are found to have 15.3 and 24.9% more absorption capacity as compared to CA nanofibers respectively. However remaining four other commercial samples (S3, S4, S5 and S6) still had nearly 50% less absorbency than pure CA nanofibers.

Although specific composition of commercial samples is not known, but from physical observation, samples S1 and S2 seem to include mainly superabsorbent polymers as their absorbent core. However S3, S4, S5 and S6 have either no or very less SPA in the combination with some fluffy cellulosic fibers. Thus the absorption in ultra-thin products (S1 and S2) is mainly due to the superabsorbent polymers in their matrix. Therefore, absorption capacity of S1 and S2 exceeds CA nanofibers when samples are immersed in DI for longer time. On the other hand, other remaining products (S3, S4, S5 and S6) have cellulosic microfiber and therefore their absorbency is found to be less than pure CA nanofibers primarily due to their lower surface area compared to CA nanofibers.

FIG. 3b summarizes the absorption capacity of all nine samples in saline solution (0.9 wt. % NaCl). In saline solution also, free absorbency of CA at time interval of 20 seconds is found to be 23.5 and 58.3% higher than CA5 and CA10 respectively. When this time interval is increased to 180 seconds, pure CA nanofibers still had about 57.1 and 69.1% higher absorbency than CA5 and CA10 samples. As can be seen from graph (FIG. 4b), the absorption capacity of CA nanofibers is more than all the commercial samples over entire time interval of test. If compared at 180 seconds, free absorbency of CA is measured to be 66.3, 56.1, 52.6, 56.6, 59.5 and 60.6% more than samples S1, S2, S3, S4, S5 and S6 respectively.

Similar trend is observed for free absorbency in synthetic urine (FIG. 3c). Absorption capacity of pure CA nanofibers for 180 seconds is 2333.1% which is 35.4 and 32.2% higher than samples SB (1506.7%) and SC (1582.1%) respectively. Similarly, pure CA nanofibers are found to have 62, 51.8, 55.6, 65.9, 54.1 and 27.9% more absorbency than S1, S2, S3, S4, S5 and S6 commercial samples respectively at the time interval of 180 seconds.

Therefore, it is very clear that in saline solution and synthetic urine, the absorption capacity of electrospun CA nanofibers is significantly higher than any of the commercial products and also to SB and SC nanofiber samples (FIGS. 3b and 3c). In case of DI water also, CA nanofibers showed large absorption capacity as compared to all samples except two commercial samples S1 and S2, which are primarily based on only SAP.

### Example 4

### Equilibrium Absorbency

Free absorbency test carried out for a time interval of 24 hours to know the maximum absorption capacity of the sample is known as equilibrium absorbency. Solutions used are distilled water, saline solution and synthetic urine. Percentage equilibrium absorbency is calculated as follows:

Q′=[(W2−W1)/W1]*100

### Where:

Q′=percent equilibrium absorbency;
W1=Initial (dry) weight of the sample; and
W2=final (wet) weight of the sample, after keeping immersed in solution for 24 hours.

Free absorbency test is extended for time interval of 24 hours in all three solutions, i.e., distilled water, saline solution and synthetic urine, to find the maximum absorption capacity, also defined as equilibrium absorbency. FIG. 3d illustrates the percentage equilibrium absorbency of electrospun samples and selected commercial samples as references. As observed, equilibrium absorbency of pure CA nanofibers is 30.7 and 60.6% more than SB and SC samples in DI water. Similarly, it is 52.5 and 65.4% higher in saline solution and 54.1 and 72.1% more in synthetic urine respectively for CA nanofibers as compared to SB and SC respectively. Therefore, it is observed that absorption capacity for CA nanofibers encapsulated with SPA (SB and SC) is less even after allowed to swell for 24 hours in all three solutions.

Furthermore while comparing the equilibrium absorbency in DI water with commercial samples, we found that absorbency of S1 and S2 is 73.3 and 28.2% higher than CA samples. This is again because of the swelling of superabsorbent polymers present in these ultra-thin products (S1 and S2) on increasing the time for immersion in DI water. However for other commercial samples (S3, S4, S5 and S6), equilibrium absorbency in DI water is 45.3, 55.1, 45.6 and 46.45% less than pure CA nanofibers samples owing to their reduced surface area.

Interestingly, the equilibrium absorbency of S1 decreases to about 65.7 and 65.5% in saline solution and synthetic urine respectively while comparing it in DI water. Similarly for S2 commercial sample, there is a decrease of 45.7 and 47.8% in saline solution and synthetic urine respectively as compared to absorbency in DI water. This behavior can be explained as follows: SPA at molecular structure contains sodium carboxylate groups on the main chain. Sodium gets detached from the chain, leaving only carboxyl ions, when it comes in contact with water [2]. This allows the sodium ions to move freely within the network, which contributes to the osmotic pressure within the gel. The mobile positive sodium ions however cannot leave the gel because they are still weakly attracted to the negative carboxylate ions along the polymer. So the driving force for swelling is the difference between the osmotic pressure inside and outside the gel. Increasing the level of sodium outside of the gel will lower the osmotic pressure and reduce the swelling capacity of the gel [16]. This swelling mechanism of SPA explains the sudden decrease in the equilibrium absorbency of commercial sanitary napkins (S1 and S2) in both saline solution and synthetic urine.

From the free absorbency and equilibrium absorbency results it can be concluded that the electrospun CA nanofibers have significantly large absorption capacity for saline solution and synthetic urine as compared to the commercial products in all the categories of use. Also, the encapsulation of SPA in these CA nanofibers (SB and SC) is decreasing the absorption capacity of nanofibers even when allowed to swell freely for 24 hours. Therefore, it is very clear that use of SPA in CA nanofibers does not facilitate in enhancing the absorption efficiency of the matrix.

### Example 5

### Absorbency Under Load (AUL):

This test is done to know the absorption capacity, if certain load is applied on the sample. By definition, this method is used to measure the ability of a superabsorbent to absorb 0.9 wt. % saline solution against certain pressure. In this study, it is used to measure the absorption capacity of elecrospun nanofibers prepared and absorbent core of commercial samples mentioned in Table I in saline solution, when compressive load is applied while absorption. The setup for AUL tester 200 as shown in FIG. 4 comprises of a glass filter plate (d=30 mm) 205, placed in petri dish 206. A filter paper (d=30 mm) 204 is placed on top of glass filter plate 205. Sample 203 cut in circular shape, with diameter of 30 mm, and weighed (W1). Weight, 50 g/cm² is kept on the assembly with help of cylindrical beaker 201 and 0.9 wt. % of NaCl solution is poured in petri dish 206. Sample is removed after 60 minutes and weighed (W2).

Percentage absorbency under load will be given by:

Q″=[(W2−W1)/W1]*100

### Where:

Q″=percentage absorbency under load;
W1=Initial (dry) weight of the sample; and
W2=final (wet) weight of the sample, after immersing in saline solution for 60 minutes.

This test measures the effect of mechanical compression on the swelling process of sample and is an important consideration for the proposed use of CA nanofibers for female hygiene applications. The compressive load applied on the sample changes the shape of the sample and may alter the surface properties like suppressing the internal structure. As a result, there is decrease in the absorbency under load compared to the free swelling i.e., equilibrium absorbency in saline solution as shown in FIG. 5. Absorbency under load for electrospun CA nanofibers was measured to be 961.9% which was reduced to 550.1 and 517.7% for CA5 and CA10 respectively. This means that CA nanofibers have 42.8 and 46.2% more absorbency under load than CA5 and CA10 respectively. Similarly absorbency under load for pure CA nanofiber was found to be 15.1, 2.2, 32.8 and 37.5% more than S1, S2, S4 and S5 samples respectively. These results also confirm that CA nanofibers exhibited much improved performance as compared to any other samples including all commercial samples.

### Example 6

### Residue Test:

This test is conducted to determine the total amount of superabsorbent material, or residue, lost from the fiber matrix after it reaches equilibrium absorption. Samples are cut into small pieces of 2×2 cm² as described in previous section. The weight of the beaker is taken as W1. Sample is kept immersed in known amount of distilled water and allowed to reach equilibrium absorbency along with the mechanical shaking for 24 hours. Sample is then removed and beaker is placed in the oven until all water evaporates. It is then weighed (W2) again in order to determine the amount of residue that remained.

Residual percentage can be determined by:

Y=[(W2−W1)/W1]*100

Where Y=residual percentage

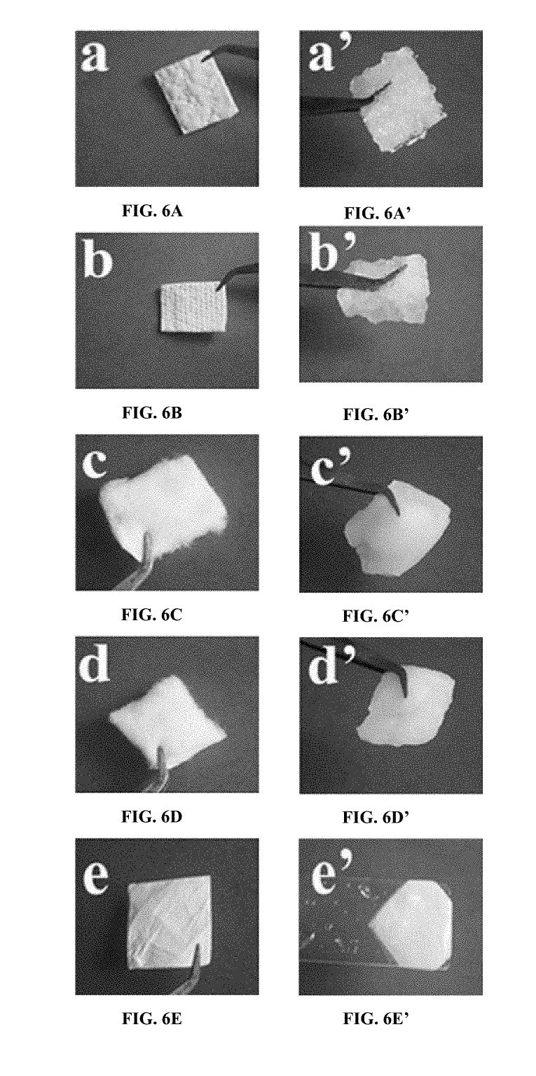

The amount of loses from the matrix is quantified by using residue test. The cellulosic fibers or loosely held SAP granules in commercial samples mainly contributes towards the residue from absorbent core. FIG. 6 represents the structure of absorbent core of commercial samples and CA nanofibers before (FIG. 6a-e) and after dipping in distilled water (FIG. 6a′-e′) for 10 minutes. SAP granules swell upon absorbing liquid and form a liquid impermeable wall of gel to inhibit further movement of liquid. Therefore, these polymers are randomly distributed within the absorbent core [2]. Smaller SAP granules increase the absorption rate because of increase in the surface area, but they have tendency to fall out of the matrix, therefore contributing towards the residue as shown in FIG. 6a′-b′. In some other cases, loosely held cellulosic fibers in the absorbent core contribute to the residue (FIG. 6c′-d′). However importantly there is no major structural change in CA nanofibers except little shrinkage (FIG. 6e′) as compared to commercial samples.

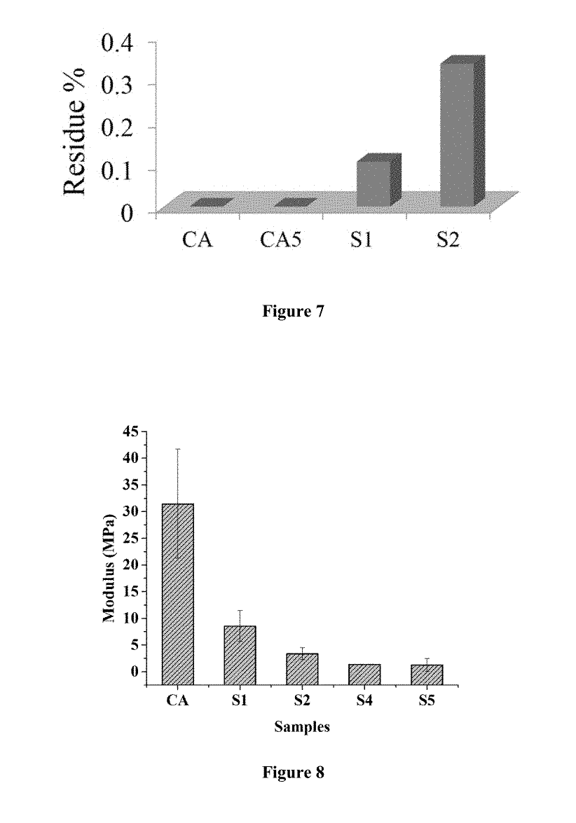

Quantitative results of residue tests are summarized in FIG. 7. Electrospun nanofibers of pure CA and with SPA (CA5) have almost negligible residue compared to the sanitary napkins, S1 (0.11%) and S2 (0.34%) respectively. For other commercial samples S3, S4, S5 and S6, whole cellulosic fiber matrix disintegrates due to mechanical shaking for 24 hours done along with equilibrium absorbency. Therefore entire initial weight of samples acts as residue and thus not compared in FIG. 7. The electrospun CA nanofibers are strongly entangled and therefore do not contribute to any residue. Same holds good after encapsulating CA nanofibres with SAP (SB and SC).

### Example 7

### Tensile Test:

Tensile test measures the force required to break a sample specimen and the extent to which the specimen stretches or elongates to that breaking point. Tensile strength is measured with Instron 5948 mechanical tester at the ambient conditions. Electrospun nanofibers mat is peeled off from the aluminum foil and cut into pieces of length 6 cm and breadth of 2 cm with thickness of approximately 0.15 mm. Similarly, the commercial samples as selected for references are cut with same dimensions with thickness varying with the sample. The sample is then placed in between pneumatic grips and the applied extension rate is 3 mm/min. Elastic modulus is then measured and compared for all the samples.

The inadequate tensile strength of absorbent core may leads to its breakage or tearing which may result in the leakage of fluid thereby decreasing the product's efficiency. Therefore, mechanical properties of electrospun CA nanofibers are measured and compared with other commercial samples. These results are represented in FIG. 8. There is a significant difference between the elastic modulus of electrospun CA nanofibers and commercial samples. The absorbent core of commercial samples (S1, S2, S4 and S5) is mainly made by loosely held cellulosic fibers and strength is provided by using different layers, above and below the core. However in case of CA nanofibers, due to compact structure and entanglement of nanofibers, modulus of elasticity is found to be 31.5±10.2 MPa. For S1, S2, S4 and S5, the modulus are 8.6±2.9, 3.4±1.1, 1.4±0.1 and 1.3±1.2 MPa respectively (FIG. 8). These results show that mechanical strength of pure CA nanofibers is more than any other commercially available samples and thus directly can be used as absorbent core in female hygiene products.

### REFERENCES

1. Garg, R.; Goyal, S.; Gupta, S. India Moves Towards Menstrual Hygiene: Subsidized Sanitary Napkins for Rural Adolescent Girls-Issues and Challenges. Maternal and Child Health Journal 2012, 16,767-774.
2. Das, D. In Composite Nonwoven Materials; Das, D., Pourdeyhimi, B., Eds.; Woodhead Publishing: United Kingdom, 2014; p 74.
3. Mohammad, J. Z.; Kourosh, K. Superabsorbent Polymer Materials: A Review. Iranian Polymer Journal 2008, 17, 451-477.
4. Kumar, K. S. Is Your Sanitary Napkin Safe, The New Indian Express, Aug. 28, 2013, Website.http://www.newindianexpress.com/cities/bangalore/Is-your-sanitary-napkin safe/2013/08/28/article1755252.ece
5. Berkley, S. F.; Hightower, A. W.; Broome, C. V.; Reingold, A. L. The Relationship of Tampon Characteristics to Menstrual Toxic Shock Syndrome. The Journal of the American Medical Association 1987, 258, 917-920.
6. Ramakrishna, S.; Fujihara, K.; Eong, T. W., Lim, T.; Ma, Z. An Introduction to Electrospinning and Nanofibers. World Scientific Publishing Co. Pte. Ltd.: Singapore, 2005.
7. Cramariuc, B.; Cramariuc, R.; Scarlet, R.; Manea, L. R.; Lupu, I. G.; Cramariuc, O. Fiber Diameter in Electrospinning Process. Journal of Electrostatics 2013, 71, 189-198.
8. Formhals, A. Process and Apparatus for Preparing Artificial Threads. U.S. Pat. No. 1,975,504, Oct. 2, 1934.
9. Doshi, J.; Reneker, D. H. Electrospinning Process and Applications of Electrospun Fibers. Journal of Electrostatics, 1995, 35, 151-160.
10. Huang, Z-M.; Zhang, Y-Z.; Kotaki, M.; Ramakrishna, S. A Review on Polymer Nanofibers by Electrospinning and Their Applications in Nanocomposites. Composites Science and Technology 2003, 63, 2223-2253.
11. Frey, M. W. Electrospinning Cellulose and Cellulose Derivatives. Polymer Reviews 2008, 48, 378-391.
12. Hubbe, M. A.; Ayoub, A.; Daystar, J. S.; Venditti, R. A.; Pawlak, J. J. Enhanced Absorbent Products Incorporating Cellulose and Its Derivatives: A Review. BioResources 2013, 8, 6556-6629.
13. Konwarh, R.; Karak, N.; Misra, M. Electrospun Cellulose Acetate Nanofibers: The Present Status and Gamut of Biotechnological Applications Biotechnology Advances 2013, 31, 421-437.
14. Zhou, W.; He, J.; Cui, S.; Gao, W. Studies of Electrospun Cellulose Acetate Nanofibrous Membranes. The Open Materials Science Journal 2011, 5, 51-55.
15. Frazier, M. Superabsorbent Nanofiber Matrices. Ph.D. Thesis, The University of Akron, Ohio, USA, 2006.
16. Elliott, M. Superabsorbent Polymers; BASF Aktiengesellschaft: Ludwigshafen Germany, 2004.

1. An eco-friendly sanitary napkin for feminine hygiene management characterized with an absorbent core having enhanced absorbancy capacity and surface area comprising an elongated absorbent body with a membrane structure composed of biocompatible polymeric nano fibers with an average diameter of 50-200 nm with an optional encapsulation.

2. The sanitary napkin absorbent core as in claim 1 wherein the biocompatible polymeric material used is cellulose acetate.

3. The sanitary napkin absorbent core as in claim 2 wherein cellulose acetate solution is obtained by dissolving cellulose acetate in a mixture of acetone and N,N-dimethylacetamide at 2:1 volume ratio.

4. The sanitary napkin absorbent core as in claim 1 wherein the average absorbency of the membrane is found to be around 1967%, 2322% and 2625% in distilled water, saline solution and synthetic urine respectively.

5. The sanitary napkin absorbent core as in claim 1 wherein the average surface area of the membrane is found to be around 50.21 m²/g

6. The sanitary napkin absorbent core as in claim 1 wherein the tensile strength of the membrane is found to be around 31.5±10.2 MPa.

7. A method of preparing an eco-friendly sanitary napkin including an absorbent core with a membrane structure comprises of biocompatible polymeric nano fibers comprising the steps of:
a. Dissolving the polymer in a mixture of acetone and N,N-dimethylacetamide at 2:1 volume ratio;
b. Obtaining the polymer solution; and
c. Electrospinning the polymer solution to obtain nano fibers to form the membrane structure.

8. The method as in claim 7 wherein the polymer used is cellulose acetate.

9. The method as in claim 8 wherein cellulose acetate solution is obtained by dissolving cellulose acetate in a mixture of acetone and N,N-dimethylacetamide at 2:1 volume ratio.
